(19) United States
(12) Patent Application Publication
(10) Pub. No.: US 2016/0059028 A1
(43) Pub. Date: Mar. 3, 2016

(54) Far infrared rays substrate applied in electric device

(71) Applicant: Chan Yuea Biotechnology CO., LTD, Taichung City (TW)

(72) Inventor: Chang-Chyun Liu, Yunlin County (TW)

(21) Appl. No.: 14/469,367

(22) Filed: Aug. 26, 2014

(51) Int. Cl. A61N 5/06

(52) U.S. Cl. CPC A61N 5/06; A61N 2005/066

(57) ABSTRACT

A far infrared rays (FIR) substrate applied in electric device mainly includes an electric device and a FIR releasing material. The electric device is operated with electric power. The electric device emits an electromagnetic wave during operation; wherein the electromagnetic wave is able to, according to Faraday law of electromagnetic induction, induce an induction electromotive force within the circuit of the electric device for emitting the electromagnetic wave outward the electric device which is able to be received by a human body. The FIR is transmitted by the electromagnetic wave to human body to generate absorption resonance by human body at 9.36 um wavelength, thus the human body is able to absorb the energy of the FIR releasing material. It is able to activate resonance of water molecular in human cells and activate metabolism.

### BACKGROUND OF THE INVENTION

1. Field of the Invention

The present invention is related to a far infrared rays (FIR) substrate applied in electric device. More particularly, the present invention is related to a FIR substrate applied in electric device which is able to generate FIR to enhance bioelectricity of human body that is able to reach deeply inside human body and activate resonance of water molecular in human cells and accelerate blood cycling.

2. Description of the Prior Art

Plants have vegetable electricity, animals have animal electricity, and human body has bioelectricity. Every variation can generate electricity. Bionics discovers that even the minus bacterium can generate electricity when it consumes glucose, and it is so-called bio electricity. Similar, metabolism and every activity of human being can generate electricity. Electrocardiography (ECG) represents waveform of heart beating, and electroencephalogram (EEG) represents waveform of brain when it acts.

Dr. Mark Bork of University of Toronto, Canada, has proven that, in mammal animal's brain, there is a structure responsible for delivering electric signal among neural cells, which is not unidirectional but mutual-directional. How does a normal human body cell generate electricity? The cell is embraced by cytochylema, and there are many charged ions inside and outside the cytoplasmic membrane, such as kalium ion, natrium ion, chlor ion, and etc, wherein the kalium ion is mainly inside the cell, and natrium ion is mainly outside the cell. In static status, these ions are relatively stable, however, when stimulation happens, membrane permeability of cytoplasmic membrane changes, and all ions become active. For example the kalium ion and natrium ion exchanges can induce bio electricity.

Bio electricity of human being is widely applied in modern medicine, such as ECG, EEG, electromyography, and electrogastrogram. These footprints are used as science evidence in diagnosis of diseases made by doctors.

Acupoints can be treated as trigger points of bio electricity, and main and collateral channels cam be treated as channels for current transmission. When a magnetic field is applied on acupoints, the velocity and current varies correspondingly, and the biocurrent is activated to generate electromagnetic waves which is then transmitted to the main an collateral channels. When it is transmitted to central nervous system, the stimulation is made for adjustment against pathological portion of human body.

According to bio magnetic theory, pathology is due to abnormal magnetic field of human body. Metabolism can reduce in various frequencies, waveforms of bio current and bio electromagnetic field. If an external magnetic field can be applied on the acupoints to compensate and adjust the disordered magnetic field inside human body, the abnormal neural activities can be balanced. The process of coordinating and suppressing exhilaration can cure and prevent diseases.

However, in modern society, cellular data communication system is widely popularized and developed to become a necessary portion of personal and business communication. Mobile phone allows user to communicate in anywhere. Besides, the functions of mobile phone are enhanced as well as the technologies of mobile phone developed. For example, now many smart phones are able to communicate, navigate, play music, and link to internet. Also, the smart phone allows user to transmit/receive email, message, and access internet by wireless communication.

Antennas of mobile phones are able to significantly influence size or area of the phone. Typically, a mobile phone has an antenna structure allowing the phone to operate in multiple operation frequencies of communication. The mobile phone is able to use various kinds of antenna, such as spiral, up-side-down F, folded dipole, and telescopic antenna. The spiral and telescopic antennas are displaced outside the mobile phone, as well as the up-side-down F and folded dipole antennas are displaced inside the mobile phone, such as shell of the mobile phone, and near the top portion thereof.

However, the disadvantages of each antenna of smart phone are, when using phone, the antenna is very close to head of user. As the antenna is close to head of user, the human body would absorb more and more radio frequency (RF) energy and radiation. The RF energy when using phone absorbed by human body is called specific absorption rate, SAR. Allowable SAR of mobile phone is regulated by government to ensure safety of users under RF energy coverage.

U.S. Pat. No. 6,741,215 disclosed an attempt to reduce exposure of mobile phone antenna radiation. The patent disclosed mobile phones with various internal and external antenna designs. These antennas can be placed near bottom of the mobile phone to reduce exposure of radiation on users. That is, by moving antenna away from user's head to protect user. Also, in other embodiments, the shell of phone is designed with obtuse angle to keep the bottom of the corner of shell away from the user's face.

Although these antenna designs reduce radiation exposure, the development of antenna, especially the internal antenna allows shrinking whole device dimension meanwhile providing relatively low SAR.

The inventor considers that the SAR of the aforementioned mobile phone can still influence bio electricity of human body and health and the inventor of the present invention was inclined to improve it, and finally succeeded in developing the substrate applied in electric device after years.

### SUMMARY OF THE INVENTION

The objective of the present invention is to provide a FIR substrate applied in electric device. The FIR substrate is able to generate miniature current stimulation by using a main frequency miniature current with the same waveform as that of the neural induced voltage, which is able to stimulate nerve fibers of warm-blooded animal like human beings, generate shock by contact acupoints, enhancing blood cycling and release tiredness.

Another objective of the present invention is to provide a FIR substrate applied in electric device. The FIR substrate is able to release FIR with emanation coefficient larger than 0.9 at 4˜14 um wavelength.

The FIR substrate being able to achieve the aforementioned objectives comprises:

an electric device, being operated with electric power, and the operation frequency and signal frequency of the electric device during operation are able to generate an electromagnetic wave, wherein the electromagnetic wave is able to, according to Faraday law of electromagnetic induction, induce an induction electromotive force within the circuit of the electric device for emitting the electromagnetic wave outward the electric device which is able to be received by a human body;

a FIR releasing material, having 70-95% Aluminum oxide and is able to release FIR with emanation coefficient larger than 0.9 at 4˜14 um wavelength, as well as being able to continually generate miniature static current at 0.06 mA the same as human body biocurrent at 0.06 mA, wherein the FIR with 4-14 um wavelength is transmitted by the electromagnetic wave to human body to generate absorption resonance with the FIR emitted by human body at 9.36 um wavelength, thus the human body is able to absorb the energy of the FIR releasing material.

### BRIEF DESCRIPTION OF THE DRAWINGS

No drawing is disclosed.

### DETAILED DESCRIPTION OF THE PREFERRED EMBODIMENT

The FIR substrate applied in electric device mainly comprises: an electric device and a FIR releasing material.

The electric device is operated by electric power. The operation frequency and signal frequency of the electric device during operation are able to generate an electromagnetic wave, wherein the electromagnetic wave is able to, according to Faraday law of electromagnetic induction, induce an induction electromotive force within the circuit of the electric device for emitting the electromagnetic wave outward the electric device which is able to be received by a human body. The electric device can be a mobile device or a smart phone (or phone). The electric device comprises: a shell, a circuit board installed in the shell (such as a PCB), circuits installed on the circuit board, such as a micro processor, memory, one or more wireless transceivers (such as cellular, WLAN, GPS, Bluetooth antenna, and etc.), audio and power circuit, and etc. People skilled in the art would know the aforementioned items. The shell, preferably, also comprises a battery for empowering the circuit. Also, the shell can comprises an audio output transmitter (such as a speaker) connected to the circuit, and one or more user input interface device (such as button or touch panel).

The FIR releasing material is set on the electric device, wherein the FIR releasing material has 70-95% Aluminum oxide and is able to release FIR with emanation coefficient larger than 0.9 at 4˜14 um wavelength, as well as being able to continually generate miniature static current at 0.06 mA the same as human body biocurrent at 0.06 mA, wherein the FIR with 4-14 um wavelength is transmitted by the electromagnetic wave to human body to generate absorption resonance with the FIR emitted by human body at 9.36 um wavelength, thus the human body is able to absorb the energy of the FIR releasing material.

The FIR with 4-14 um wavelength is able to improve growth of animals and plants. The FIR would not upset human body, and is able to enhance biocurrent of human body, which is characterized in that the FIR is able to reach deeply into human tissue, enhance resonance of water molecular inside human cells, to enrich vitality of cells, accelerate blood cycling, activate metabolism, enhance regeneration of tissues, and activate immunity system. So far, it is medically proven that FIR is able to cure many diseases.

According to the aforementioned ideas, the FIR releasing material comprises at least one of aluminum oxide, titanium dioxide, silicon oxide, magnesium oxide, zirconium oxide, and zinc oxide.

In the embodiment, the FIR material is a high efficient FIR ceramic powder, which is proven by many experiments that it's effects is practical. The material is composed of many natural minerals comprising 70˜95% aluminum oxide, 1˜20% silicon oxide, and 1˜10% zirconium oxide. Also it may comprise titanium dioxide or more natural minerals, such as silicon oxide and zinc oxide. Referring to black body and being measured by FIR spectroscope, the FIR releasing material is able to release FIR with emanation coefficient larger than 0.98 at 4˜14 um wavelength. Based on US AATCC100 standard test method of antimicrobial evaluation testing, the released FIR is able to reach 99.9% up antimicrobial rate against staphylococcus aureus and colibacillus.

FIR is with a certain wavelength of 4˜1000 um, and the FIR emitted from human body is usually with wavelength 414 um. The light waves with the same wavelength are able to be superpositioned. The FIR with the same wavelength as FIR emitted from human body is able to enhance health of human body, and the FIR with wavelength 4˜14 um is the same as FIR emitted from human body, which is easily to be absorbed by human body and transformed into biocurrent inside human body that can activate water molecular generating frictional heat and “absorption resonance”. When the FIR with wavelength 4˜14 um induces the “absorption resonance” in human body, it can penetrate deeply to 3˜5 mm under skin, thus the water molecular inside cells is able to be resonated to generate internal energy, which can accelerate blood cycling, activate cell tissue, enhance metabolism, and achieve health care.

Since FIR is a kind of radiation heat, the energy emitting party and the energy receiving party has to be with the same wavelength for generating radiation resonance. The FIR releasing material of the present invention is characterized in high emanation coefficient, which is able to emit FIR with wavelength 4˜14 um and emanation coefficient larger than 0.9 under normal temperature. It reaches peak radiation wavelength to 9.1 um at 45 Celsius degree, which is similar to wavelength of human radiation FIR. The FIR releasing material is heated up to activate FIR.

The present invention is able to enhance biocurrent of human body of user who use electromagnetic product. The present invention applied microscope and image process to observe users. One user used computer normally and used computer after the FIR substrate is applied, meanwhile another nineteen users used smart phone normally and used smart phone after the FIR substrate is applied. The variation of peripheral cycling blood rate (capillary) of these users is observed to verify effects of the FIR substrate.

One experimental user is measured to set up a reference value of his blood flow rate before the user uses computer. Then the user starts to operate a computer, and his blood flow rate is measured twice by every 30 minutes. Then the FIR substrate is attached to the power supply peripheral area behind the computer monitor, and the user's blood flow rate is then measured twice by every 30 minutes. Five sets of data are retrieved for later comparison.

Nineteen experimental users are required to use smart phone (or phone) 5 minutes. Then the users held smart phone (or phone) in right hand while the left hand is treated with first time miniature cycling measurement to measure blood flow rate of capillary of middle finger. Then the used smart phone (or phone) is attached with the FIR substrate for the users to continually use the smart phone (or phone) 5 minutes and the blood flow rate is measured again with the same method after the FIR substrate is applied. The present invention applies reflective microscope, and it is suitable in observing miniature cycling microscope image by applying baby body oil with proper reflection index on desired portion of body and illuminating the desired portion with proper light. Then the retrieved image is post processed by optical flow method to calculate blood flow rate. The nineteen users who took the smart phone (or phone) experiment of using FIR substrate, their blood flow rate are all higher than that before using the FIR substrate. Results show that the blood flow rate increases between 6%-44% in Table 1.

| | P1 | P2 | P3 | P4 | P5 | P6 | P7 | P8 | P9 |
|---|---|---|---|---|---|---|---|---|---|
| Before | 65.96 | 12.89 | 87.64 | 60.61 | 163.13 | 9.08 | 94.35 | 66.49 | 39.46 |
| After | 73.04 | 17.66 | 102.37 | 66.49 | 172.26 | 13.1 | 115.66 | 80.52 | 48.30 |
| Percentage Diff. | +11% | +37% | +17% | +10% | +6% | +44% | +23% | +21% | +22% |

| | P10 | P11 | P12 | P13 | P14 | P15 | P16 | P17 | P18 | P19 |
|---|---|---|---|---|---|---|---|---|---|---|
| Before | 74.60 | 69.79 | 18.84 | 78.57 | 34.40 | 44.68 | 75.88 | 70.67 | 46.84 | 86.07 |
| After | 90.52 | 84.05 | 25.80 | 91.93 | 41.65 | 52.27 | 86.97 | 86.00 | 55.11 | 96.86 |
| Percentage Diff. | +21% | +20% | +37% | +17% | +21% | +17% | +15% | +22% | +18% | +13% |

Table 1 testing result of peripheral miniature cycling of users using smart phone (or phone) and FIR substrate

The testing result of blood flow of experimental users before using computer and after using computer with FIR substrate attached on the monitor power peripheral area are shown in Table 2, wherein the measuring unit is pixel/frame. The experimental users, after using computer 30 minutes, present that the blood flow rate is slower by 40.4% referring to natural status. Moreover, the second measurement shows that the blood flow rate is further decreased by 6.8%. After the FIR substrate is applied, the blood flow rate is slightly increased by 0.4% at first measurement, and the measured value is increased by 19.4% at second measurement, which is shown in Table 3.

TABLE 2
testing results of peripheral cycling of users using computer and FIR
substrate

| | P1_Natural | 1^{st} Measure | 2^{nd} Measure |
|---|---|---|---|
| Before | 8.86 | 5.29 | 4.93 |
| After | | 4.95 | 5.91 |

Table 2 testing result of peripheral cycling of users using computer and FIR substrate

TABLE 3
testing results of peripheral cycling of users using computer and FIR
substrate

| | Natural & | Before 1^{st} & | Before 2^{nd} & | After 1^{st} & |
|---|---|---|---|---|
| | Before 1^{st} | Before 2^{nd} | After 1^{st} | After 2^{nd} |
| Percentage Diff. | −40.4% | −6.8% | +0.4% | +19.4% |

Table 3 testing result of peripheral cycling of users using computer and FIR substrate

Testing result of the all nineteen experimental users who use smart phone (or phone) with FIR substrate shows that the blood flow rate increased positively, which means that the FIR substrate applied in smart phone (or phone) is able to effectively improve peripheral blood flow rate of human body. Only that the effect varies among users since each experimental user is different in blood flow rate status. Some groups present faster blood flow rate, while some groups present slower blood flow rate, which causes variation range of 38% of blood flow rate amplitude. Besides, when the FIR substrate is applied in computer, the blood flow measured value is different from which under normal situation and presents improvement comparing with no use of the FIR substrate. It is noted that, after using the FIR substrate 60 minutes, the blood flow rate can increase 19.4%.

From perspective of miniature cycling, blood carries nutrition for body tissues and cells. If the miniature cycling is lack of sufficient blood supply, people would feel weak, tired, stiff, and etc. In long term observation, it would lead to serious diseases. The present invention is proven that the smart phone (or phone) or computer monitor with FIR substrate is able to improve miniature cycling.

As aforementioned, the present invention is novel in technology and advantaged in many effects that the prior arts lack. The present invention conforms to the novelty and non-obviousness of patentability. Please the examiner carefully considering the application of the present invention and allowing the application.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A far infrared rays (FIR) substrate applied in electric device, comprising:
an electric device, being operated with electric power, and the operation frequency and signal frequency of the electric device during operation are able to generate an electromagnetic wave, wherein the electromagnetic wave is able to, according to Faraday law of electromagnetic induction, induce an induction electromotive force within the circuit of the electric device for emitting the electromagnetic wave outward the electric device which is able to be received by a human body;
a FIR releasing material, having 70-95% Aluminum oxide and is able to release FIR with emanation coefficient larger than 0.9 at 4˜14 um wavelength, as well as being able to continually generate miniature static current at 0.06 mA the same as human body biocurrent at 0.06 mA, wherein the FIR with 4-14 um wavelength is transmitted by the electromagnetic wave to human body to generate absorption resonance with the FIR emitted by human body at 9.36 um wavelength, thus the human body is able to absorb the energy of the FIR releasing material.
